(19) United States
(12) Patent Application Publication
(10) Pub. No.: US 2024/0423801 A1
(43) Pub. Date: Dec. 26, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Seung-Beom Yi, Mission Viejo, CA (US); Grant Matthew Stearns, Costa Mesa, CA (US); Lyon Huynh Ly, Anaheim, CA (US); Rhayoung Kwon, Irvine, CA (US); Thanh Huy Le, Oceanside, CA (US); Amanda Tjipta Sugijoto, Tustin, CA (US); Eric Michael Oberwise, Newport Beach, CA (US)

(21) Appl. No.: 18/821,257

(22) Filed: Aug. 30, 2024

Related U.S. Application Data
(63) Continuation of application No. PCT/US2023/017089, filed on Mar. 31, 2023.
(60) Provisional application No. 63/327,121, filed on Apr. 4, 2022.

(51) Int. Cl. A61F 2/24

(52) U.S. Cl. CPC A61F 2/2466; A61F 2/2454; A61F 2210/0076; A61F 2220/0016

(57) ABSTRACT

An implantable device or implant is configured to be positioned within a native heart valve to allow the native heart valve to form a more effective seal. The device can be deployed from a delivery system including one or more lines which can be decoupled from the device by energizing. The device can include contoured clasps which approximate the shape of the native valve when the clasps are in the closed position. The device can include a coaptation element with body portion including a plurality of struts and openings defining a lattice structure. One or more surfaces of one or more components of the device can be coated to improve the visibility of the device during deployment and implantation.

### RELATED APPLICATIONS

The present application is a continuation of Patent Cooperation Treaty application no. PCT/US2023/017089, filed on Mar. 31, 2023, which claims the benefit of U.S. Provisional patent application No. 63/327,121, filed on Apr. 4, 2022, which are both incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

In some implementations, a valve repair device for repairing a native valve of a patient includes a pair of paddles and a pair of contoured clasps moveable between an open position and a closed position. The contoured clasps approximate the shape of the native valve when the valve repair device is deployed in the native valve and the contoured clasp is in the closed position.

In some implementations, each contoured clasp includes a contoured moveable arm and a fixed arm.

In some implementations, the fixed arm has a contour which mirrors the contoured moveable arm.

In some implementations, the contoured clasp includes one or more barbs.

In some implementations, each paddle includes an inner paddle and an outer paddle.

In some implementations, the device incudes a coaptation element disposed between the contoured clasps.

In some implementations, the coaptation element includes a plurality of struts and a plurality of openings defining a lattice structure.

In some implementations, one or more components of the device includes a coating which decreases a number of waveforms which echo back when using an imaging technology.

In some implementations, a system includes the valve repair device coupled to one or more catheters.

In some implementations, the valve repair device and/or one or more of the catheters are sterilized.

In some implementations, a valve repair device for repairing a native valve of a patient includes an anchor portion with a pair of anchors and a coaptation portion including a coaptation element. The coaptation element includes a body portion with a plurality of struts and a plurality of openings defining a lattice structure.

In some implementations, the lattice structure defines wing portions of the body portion of the coaptation element.

In some implementations, the lattice structure defines substantially an entirety of the body portion of the coaptation element.

In some implementations, the struts compress when subjected to a force applied to the body portion of the coaptation element.

In some implementations, the struts return to an uncompressed position when the force is removed from the body portion of the coaptation element.

In some implementations, clasps of the anchor portion approximate a shape of a mitral valve when the valve repair device is deployed in the native valve and the contoured clasp is in a closed position.

In some implementations, the device includes a coating which decreases the number of waveforms which echo back when using an imaging technology.

In some implementations, a system includes the valve repair device coupled to one or more catheters.

In some implementations, the valve repair device and/or one or more of the catheters are sterilized.

In some implementations, a method for implanting a valve repair device for repairing a native valve of a patient includes the steps of deploying the valve repair device from a delivery system in a native heart of the patient, actuating the valve repair device with an actuation element, applying a tension force to an actuation wire coupled to an actuation loop of the valve repair device to open a clasp of the device, lessening the tension force to close the clasp to engage the native valve, energizing an electrode at a distal end of the actuation wire to decouple the actuation wire from the actuation loop, and retracting the actuation wires and the delivery system from the native heart.

In some implementations, the actuation wire includes an insulation.

In some implementations, the actuation wire includes a protector.

In some implementations, the electrode is energized by an energy source. In some implementations, a handle controls a flow of energy to the electrode.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In some implementations, one or more of the valve repair device, the delivery system, the actuation element, the actuation wire, the actuation loop, the clasps, and the electrode are sterilized.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only examples of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIG. 25A illustrates an example valve repair device with paddles in a closed position;

FIG. 25B illustrates a top view of an example valve repair device;

FIG. 26 illustrates a perspective view of an example implantable device having paddles of adjustable widths;

FIG. 27 is a cross-section of the implantable device of FIG. 26 in which the implantable device is bisected;

FIG. 28 is a cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of the connector of the implantable device and coupled to a paddle width control;

FIG. 31 is a schematic cross-section illustration of the geometry of mitral valve leaflets;

FIG. 32 illustrates an example valve repair device with paddles in a closed position;

FIG. 33 shows a side view of an example implantable device or implant in a closed position;

FIG. 34 shows a side view of an example implantable device or implant in an extended position with clasps closed;

FIG. 35 shows a side view of the implantable device or implant of FIG. 34 with the clasps in an open position;

FIGS. 36-37 show an example of an implantable device or implant in various stages of deployment;

FIG. 38-40 show an example of an implantable device or implant, in various stages of deployment;

FIG. 41A illustrates a schematic view of an example deployment system for an implantable device or implant;

FIG. 41B illustrates a schematic view of an example deployment system for an implantable device or implant;

FIG. 42A illustrates a schematic view of an example deployment system for an implantable device or implant;

FIG. 42B illustrates a schematic view of an example deployment system for an implantable device or implant;

FIG. 43A illustrates a schematic view of an example deployment system for an implantable device or implant;

FIG. 43B illustrates a schematic view of an example deployment system for an implantable device or implant;

FIG. 44A illustrates a schematic view of an example deployment system for an implantable device or implant;

FIG. 44B illustrates a schematic view of an example deployment system for an implantable device or implant;

FIG. 45 illustrates a perspective view of an example spacer or coaptation element for an implantable device or implant;

FIGS. 46-48 show various views of an implantable device or implant incorporating the spacer or coaptation element of FIG. 45;

FIG. 49 illustrates a schematic view of an example method of treating a component of an implantable device or implant; and

FIG. 50 shows the surface of a component of an implantable device or implant after the treatment method of FIG. 49.

### DETAILED DESCRIPTION

The following description refers to the accompanying drawings, which illustrate examples of the present disclosure. Some implementations having different structures and operation do not depart from the scope of the present disclosure.

Examples of the present disclosure are directed to systems, devices, methods, etc. for repairing a defective heart valve. For example, implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

The treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

The term “simulation” encompasses use on a cadaver, computer simulator, imaginary person (e.g., if they are just demonstrating in the air on an imaginary heart), etc.

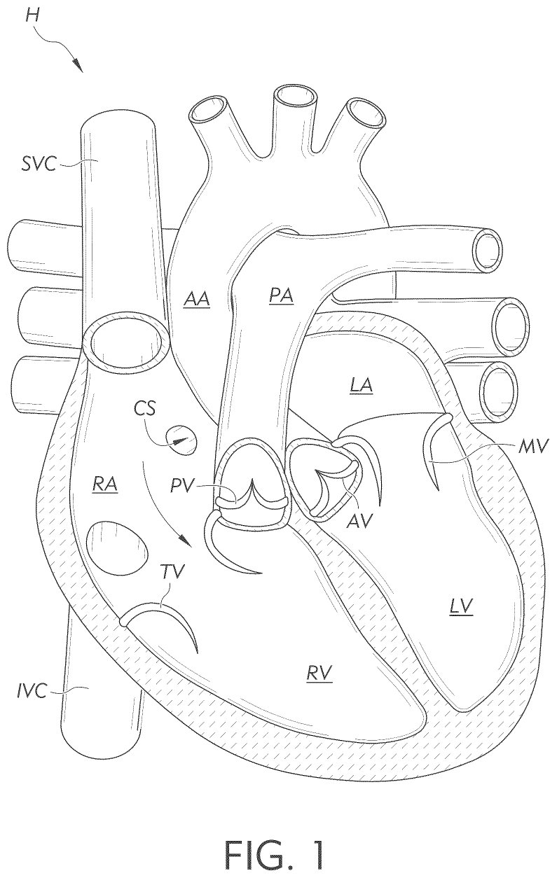

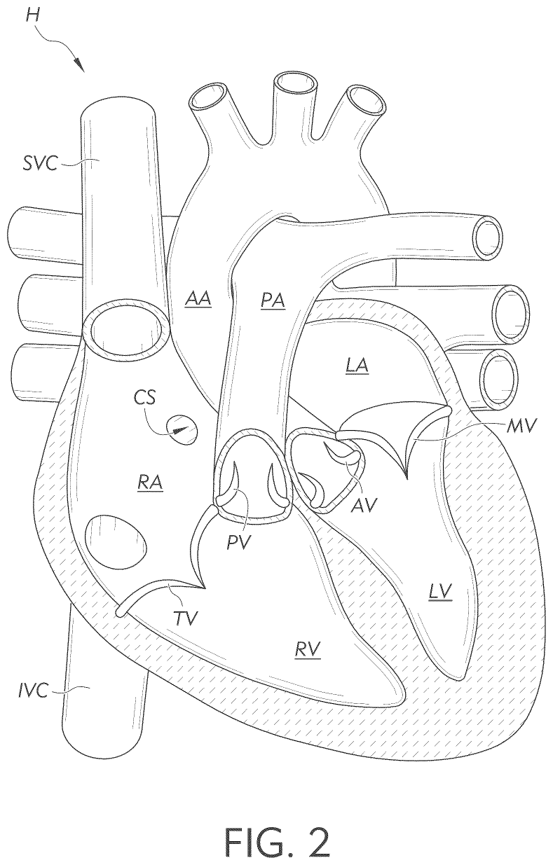

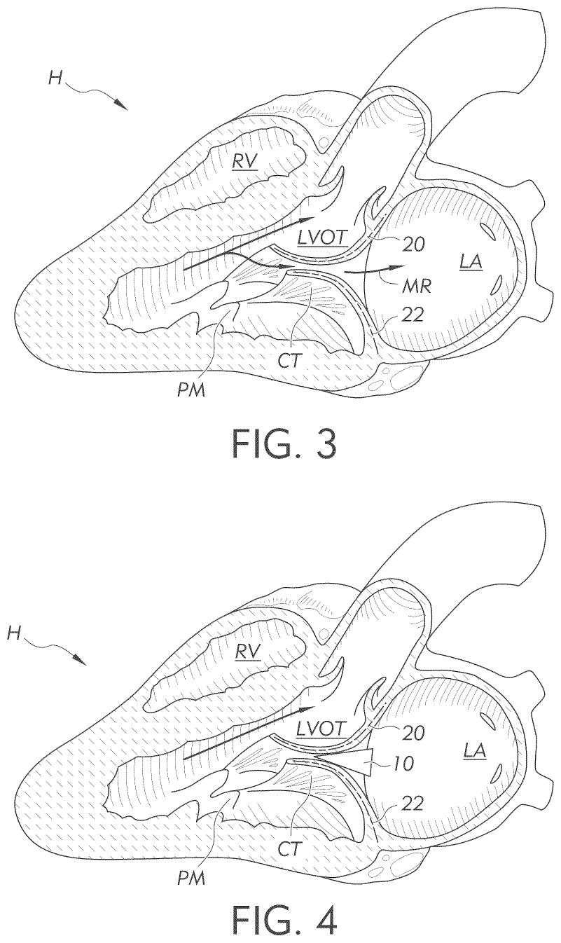

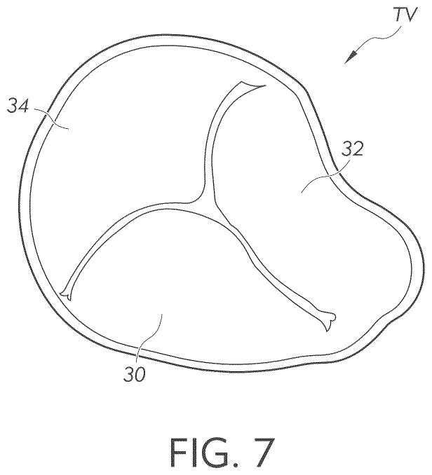

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

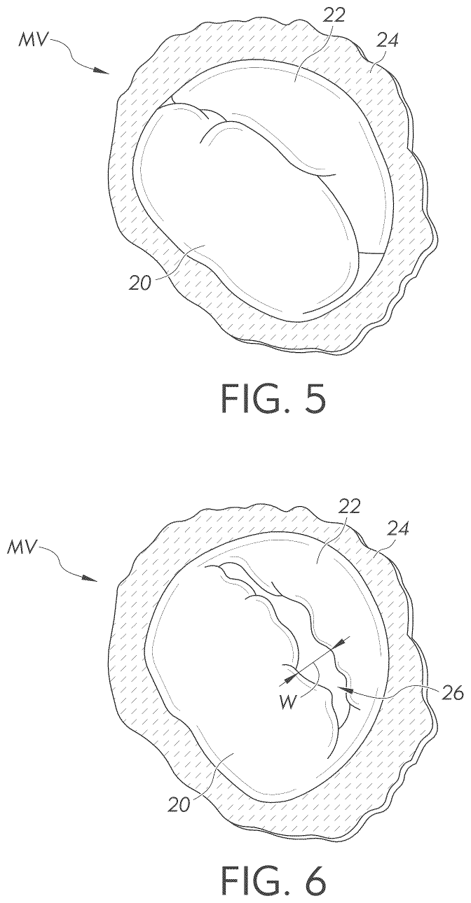

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes may impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

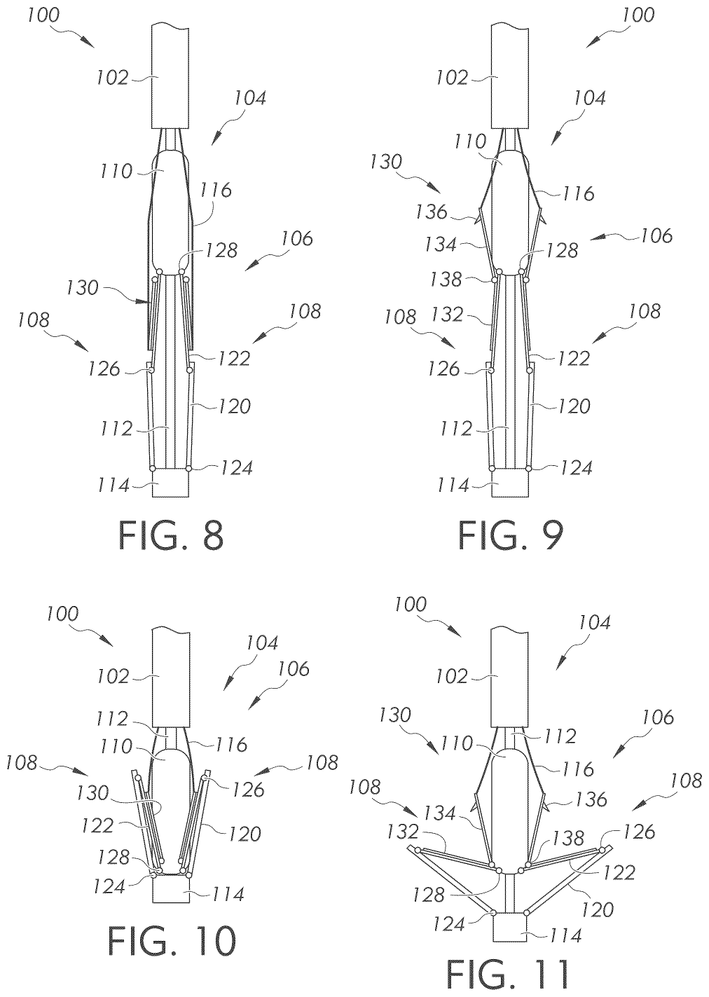

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and an optional coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

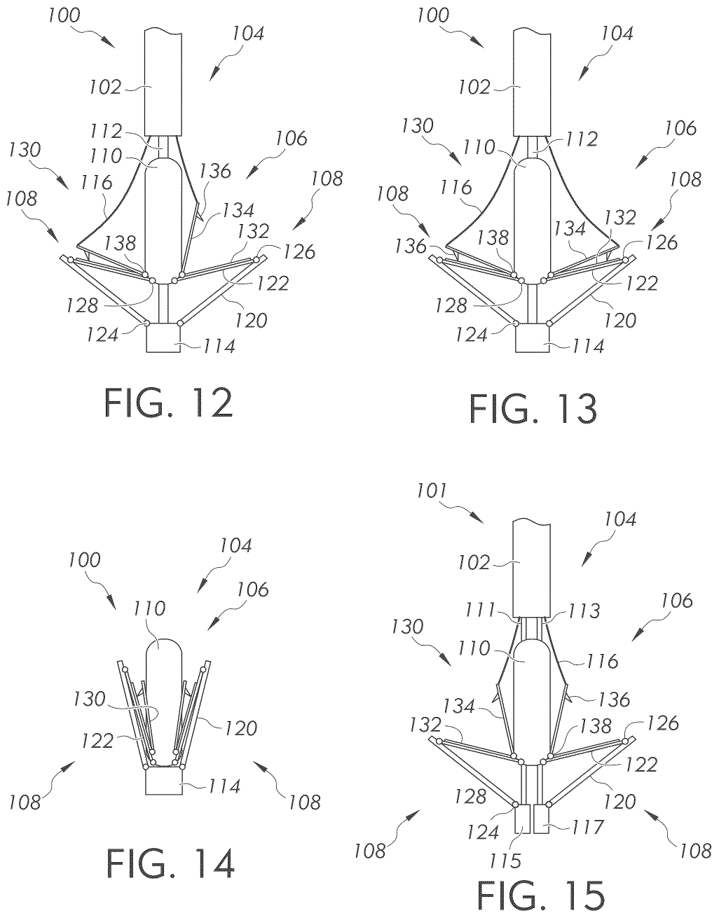

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder-and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

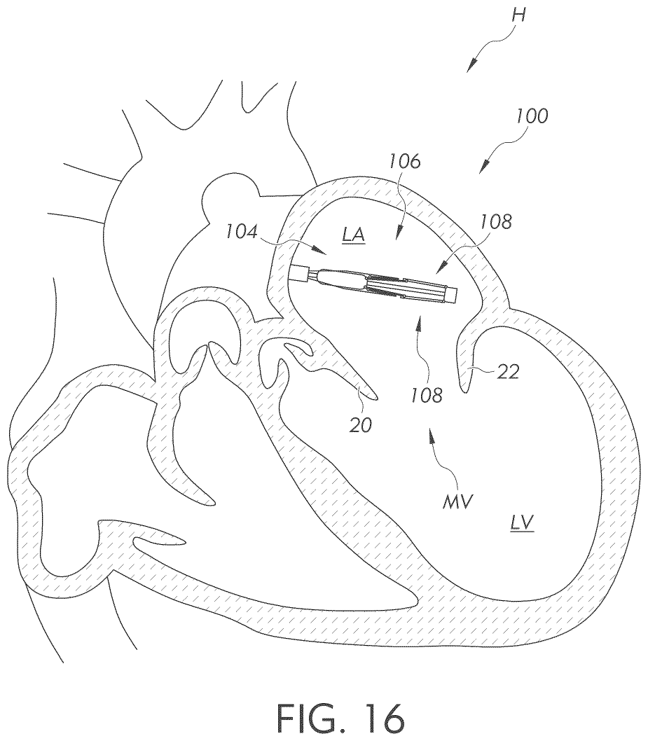

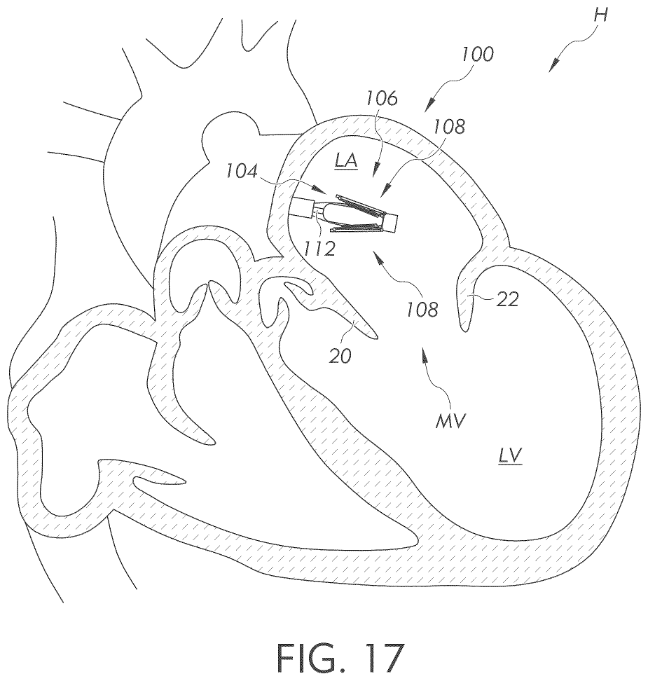

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

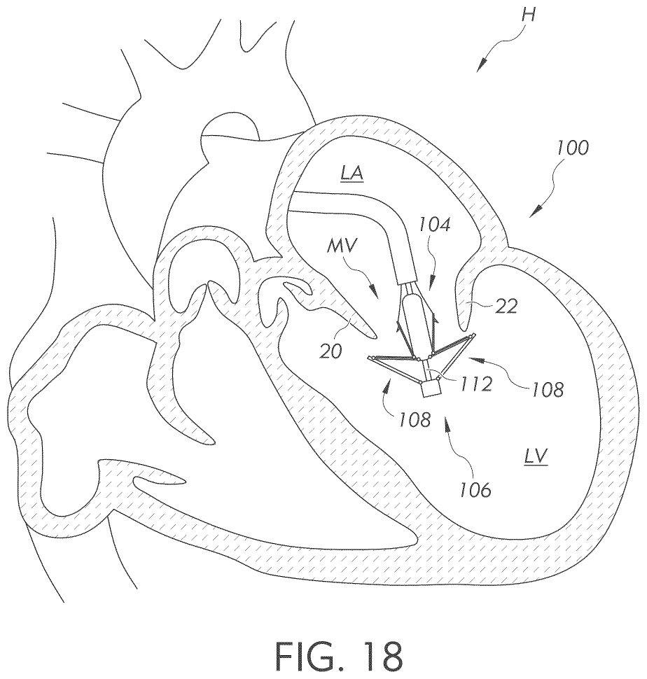

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

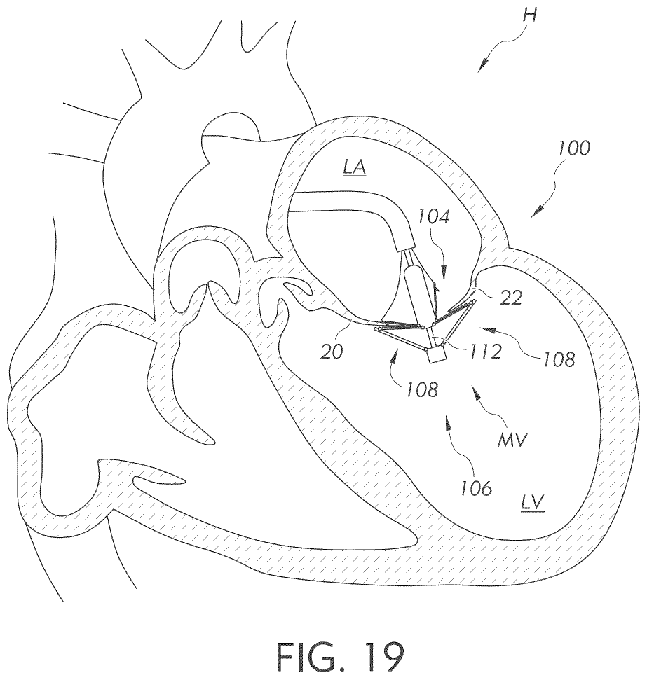

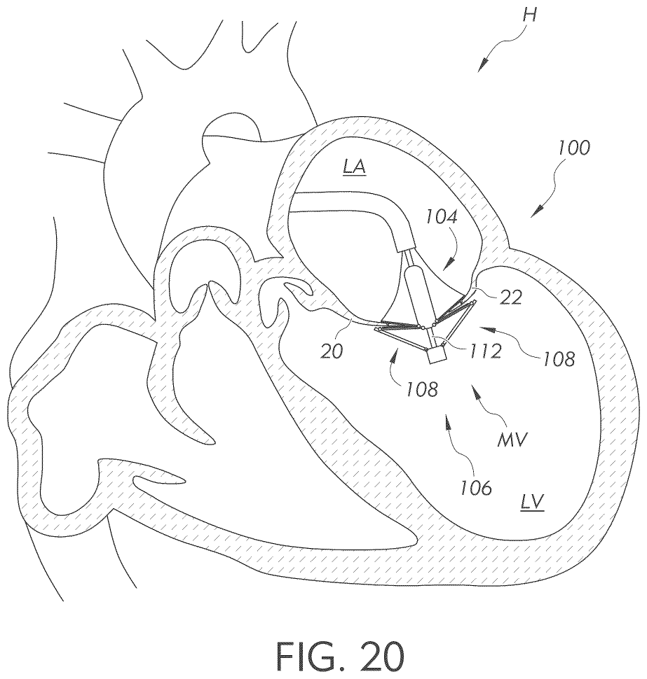

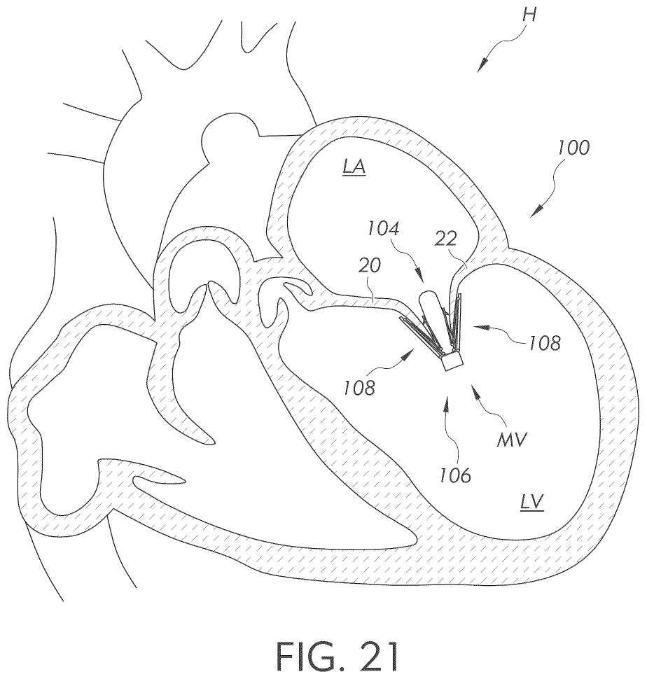

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

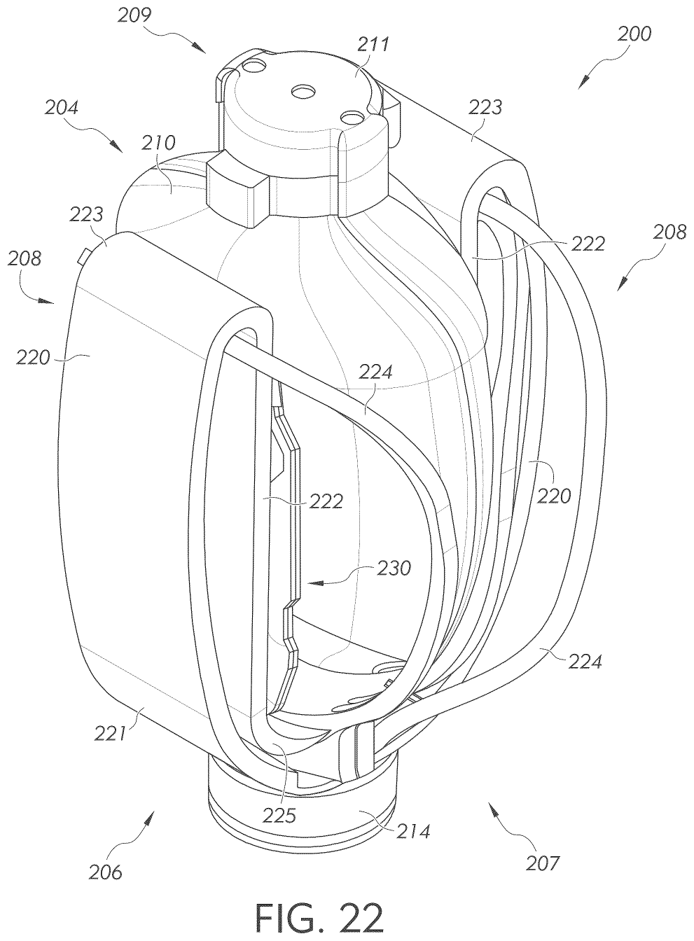

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes an optional coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism (e.g., coupler, clamp, tether, etc.) of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., plate, bar, sheet, etc.) or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

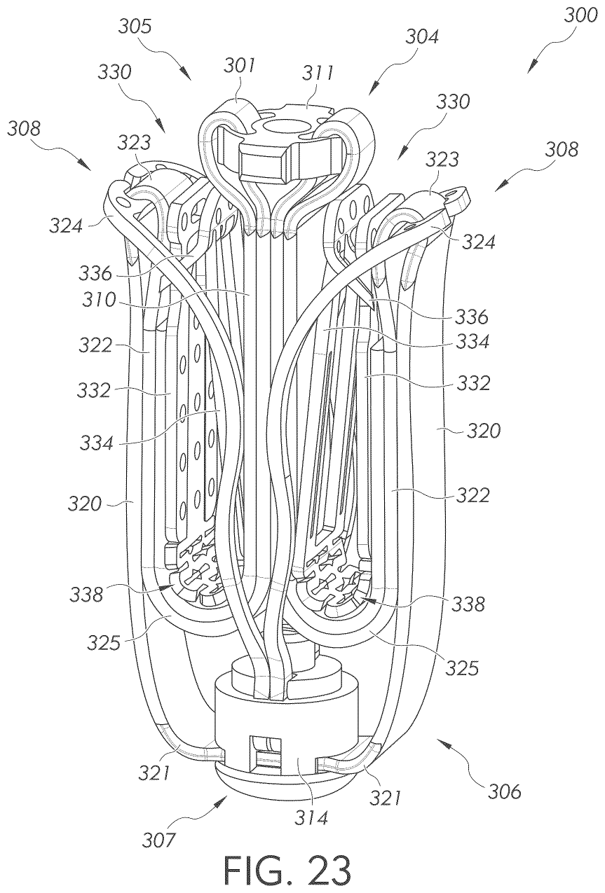

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes an optional coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., coupler, clamp, tether, etc.) of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with an optional coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

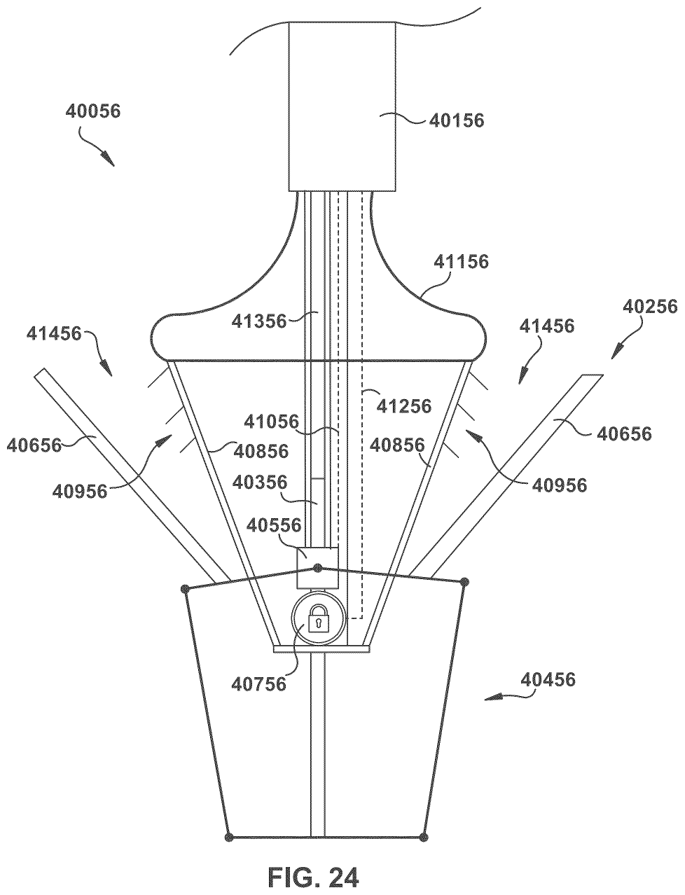

FIG. 24 illustrates an example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856 (e.g., clasps, gripping arms, etc.). In one example, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include an optional barbed portion 40956 for attaching the gripping members to valve tissue when the valve repair device 40256 is attached to the valve tissue. When the paddles 40656 are in the closed position, the paddles engage the gripping members 40856, such that, when valve tissue is attached to the optional barbed portion 40956 of the gripping members, the paddles secure the valve repair device 40256 to the valve tissue. In some implementations, the gripping members 40856 are configured to engage the paddles 40656 such that the optional barbed portion 40956 engages the valve tissue and the paddles 40656 to secure the valve repair device 40256 to the valve tissue. For example, in certain situations, it can be advantageous to have the paddles 40656 maintain an open position and have the gripping members 40856 move outward toward the paddles 40656 to engage valve tissue and the paddles 40656.

While the example shown in FIG. 24 illustrates a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056, a gripper control mechanism 41156, and a lock control mechanism 41256. The paddle control mechanism 41056 is mechanically attached to the coupler 40556 to move the coupler along the shaft, which causes the paddles 40656 to move between the open and closed positions. The paddle control mechanism 41056 can take any suitable form, such as, for example, a shaft or rod. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture or wire, a rod, a catheter, etc.

The lock control mechanism 41256 is configured to lock and unlock the lock. The lock 40756 locks the coupler 40556 in a stationary position with respect to the shaft 40356 and can take a wide variety of different forms and the type of lock control mechanism 41256 can be dictated by the type of lock used. In examples in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position.

The valve repair device 40256 is movable from an open position to a closed position. The base assembly 40456 includes links that are moved by the coupler 40556. The coupler 40556 is movably attached to the shaft 40356. In order to move the valve repair device from the open position to the closed position, the coupler 40556 is moved along the shaft 40356, which moves the links.

The gripper control mechanism 41156 is moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripping members 40856. When the line(s) is pulled, the gripping members 40856 move inward, which causes the opening 41456 between the gripping members and the paddles 40656 to become wider.

In order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

After the paddles 40656 are moved to the closed position, the lock 40756 is moved to the locked condition by the locking control mechanism 41256 to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 is disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

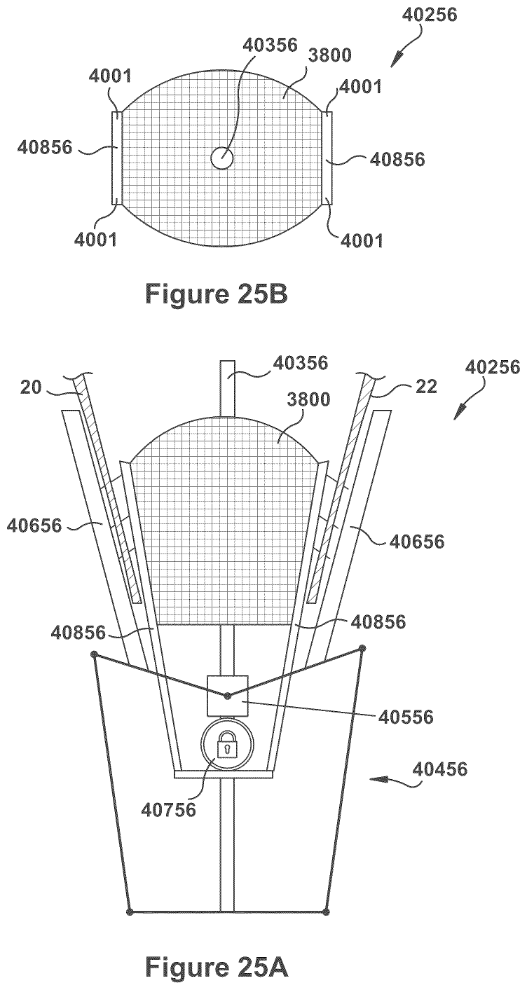

Referring to FIGS. 25A-25B, an example implementation of a valve repair device 40256 has an optional coaptation element 3800. The valve repair device 40256 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 40656 to adjust the size of the coaptation element. In the example illustrated by FIGS. 25A and 25B, the size of the coaptation element 3800 can be reduced by squeezing the coaptation element with the paddles 40656 and can be increased by moving the paddles 40656 away from one another. The coaptation element 3800 can extend past outer edges 4001 of the gripping members or clasps 40856 as illustrated for providing additional surface area for closing the gap of a mitral valve.

The coaptation element 3800 can be coupled to the valve repair device 40256 in a variety of different ways. For example, the coaptation element 3800 can be fixed to the shaft 40356, can be slidably disposed around the shaft, can be connected to the coupler 40556, can be connected to the lock 40756, and/or can be connected to a central portion of the clasps or gripping members 40856. In some implementations, the coupler 40556 can take the form of the coaptation element 3800. That is, a single element can be used as the coupler 40556 coupler that causes the paddles 40656 to move between the open and closed positions and the coaptation element 3800 that closes the gap between the leaflets 20, 22 when the valve repair device 40256 is attached to the leaflets.

The coaptation element 3800 can be disposed around one or more of the shafts, rods, tubes, or other control elements of the valve repair system 40056. For example, the coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

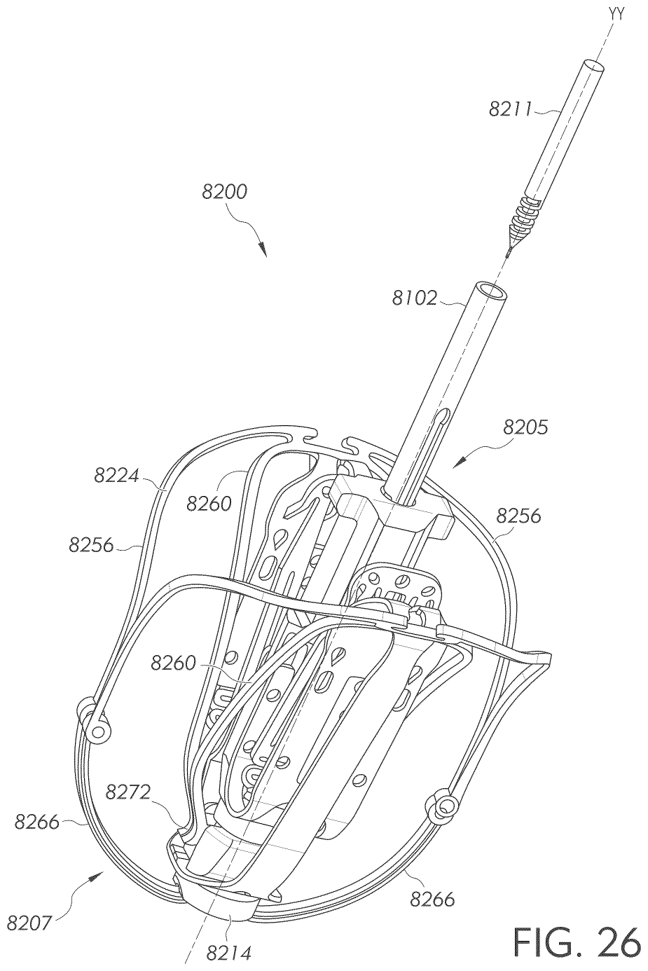

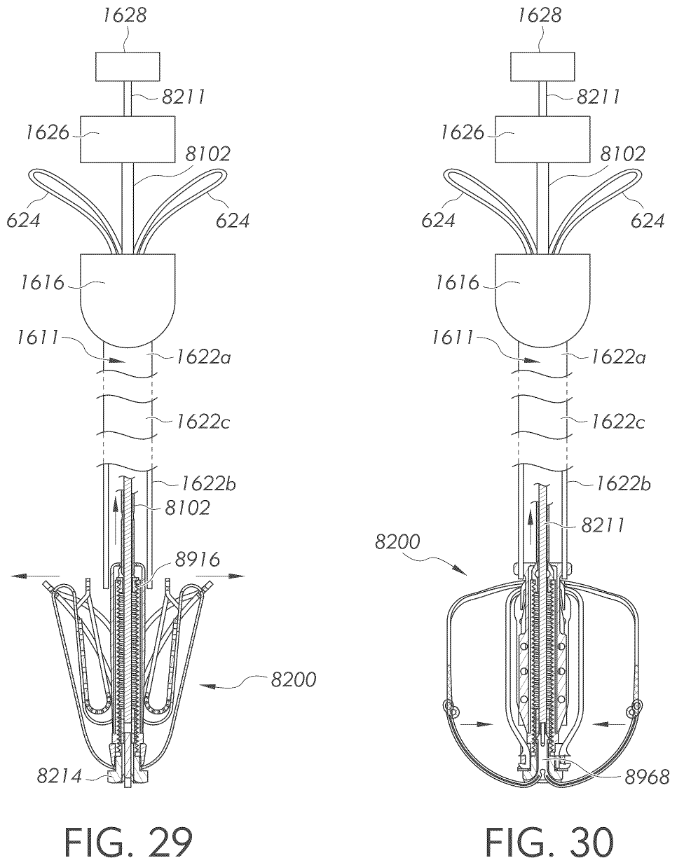

FIGS. 26-30 illustrate an example of one of the many valve repair systems for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the implantable device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

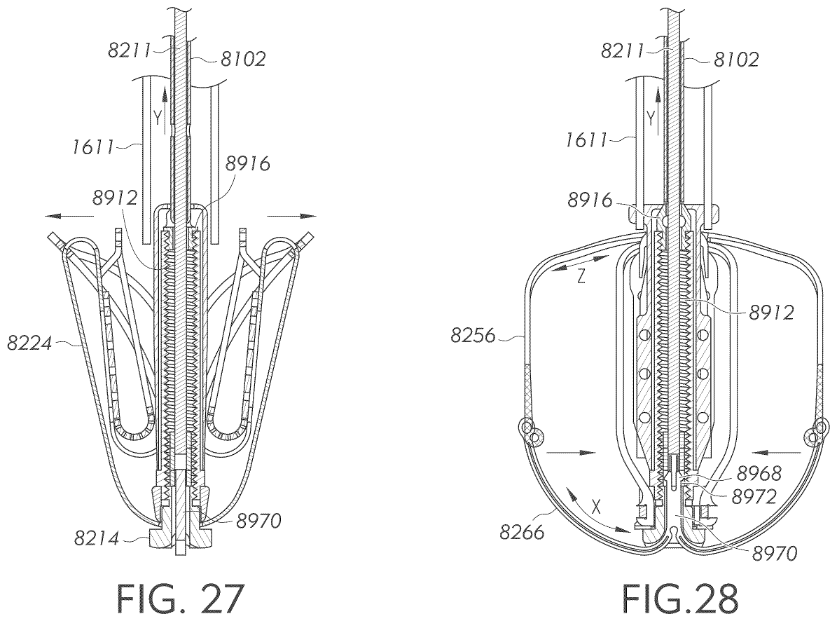

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as the other examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the implantable device 8200. The actuation element 8102 can extend distally from the paddle actuation control 1626, through the handle 1616, through the delivery shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

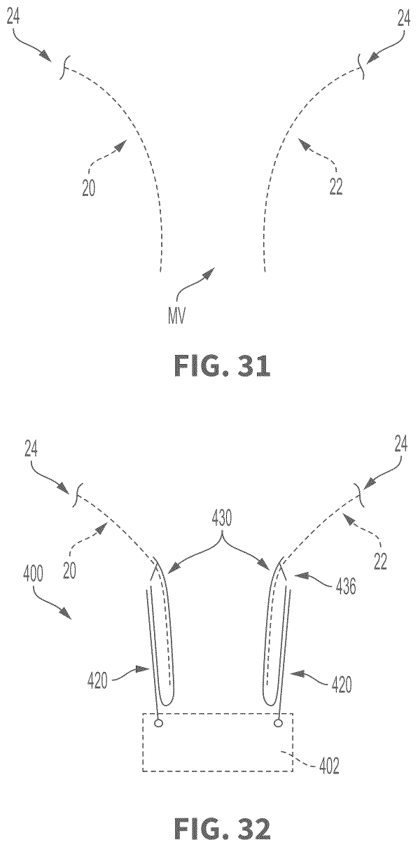

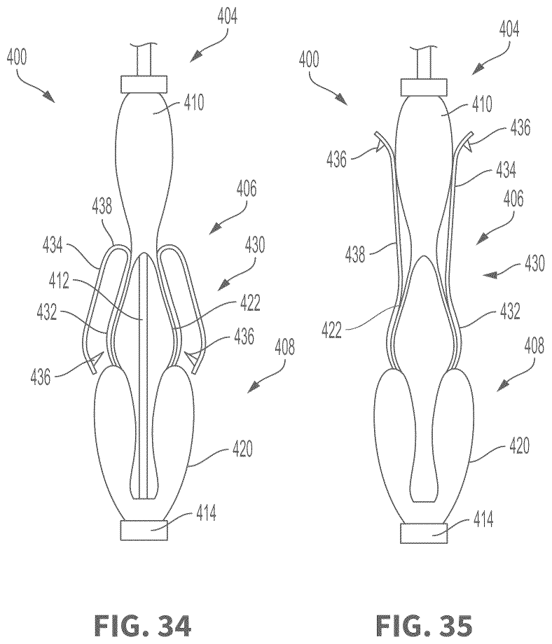

Referring to FIGS. 31-35, a schematically illustrated implantable or valve repair device 400 (e.g., an implantable prosthetic device, a prosthetic spacer device, a valve repair device, etc.) is shown in various stages of deployment. The device or implant 400 can include any other features for an implantable device, implant, or valve repair device discussed in the present application or the applications cited above, and the device 400 can be positioned to engage valve tissue (e.g., leaflets 20, 22, 30, 32, 34) as part of any suitable valve repair system (e.g., any valve repair system disclosed in the present application or applications cited above). The device 400 can be sized, shaped, or otherwise configured to approximate the shape and geometry of the leaflets of a native valve (e.g., a native mitral valve, native tricuspid valve, etc.) when the device 400 is implanted, such as when the device 400 engages the leaflets 20, 22, 30, 32, 34.

FIG. 31 illustrates a side-schematic view of the two leaflets 20, 22 of the mitral valve MV. As described above, the leaflets 20, 22 extend radially inwardly from the annulus 24 which encircles the leaflets 20, 22. As shown, the leaflets 20, 22 form a tapered-or funnel-shaped opening descending from the annulus 24. Extending radially inwardly from the annulus 24, the leaflets 20, 22 curve downwardly at an increasingly greater angle toward the opening of the mitral valve MV. As such, the distance between the leaflets 20, 22 gradually decreases from the annulus 24 to the opening, forming a curved or contoured shape.

Referring to FIGS. 32-35, the implantable device or valve repair device 400 can be configured in a variety of ways such that, when the device 400 engages the leaflets 20, 22 of the mitral valve MV, one or more contours of the device 400 conform to the shape and geometry of the leaflets 20, 22. For example, attachment portions or gripping members, clasps, coaptation element, and/or paddles of the device 400 can be curved, contoured, or otherwise shaped to approximate the shape and geometry of the native leaflets 20, 22 in the mitral valve MV.

As shown in FIG. 32, in one implementation, the device 400 is a valve repair device configured to approximate the shape and geometry of the leaflets 20, 22 of the mitral valve when the device 400 is implanted in the mitral valve. The device 400 can include any features of any valve repair devices previously described herein, such as the valve repair device 40256 described in FIG. 24 or the valve repair device 40256 of FIGS. 25A-25B, and the device 400 can be positioned to engage valve tissue 20, 22 as part of any suitable valve repair system (e.g., any valve repair system disclosed in the present application).

The device 400 includes a base 402, a pair of paddles 420, and a pair of clasps or gripping members 430. In one example, the paddles 420 can be integrally formed with the base 402. For example, the paddles 420 can be formed as extensions of links or other portions of the base 402. In some implementations, the gripping members 430 are pivotally connected to the base 402, such that the gripping members 430 can be moved to adjust the width of the opening between the paddles 420 and the gripping members 430. Each gripping member 430 can include barbs, friction-enhancing elements, or other securing structures 436 (e.g., protrusions, ridges, grooves, textured surfaces, adhesive, etc.) for attaching the gripping members 430 to engage the valve tissue (e.g., leaflets 20, 22) when the device 400 is attached to the valve tissue. When the paddles 420 are in the closed position, the paddles 420 engage the gripping members 430 such that, when valve tissue is attached to the gripping members 430, the paddles 420 secure the relative position of the leaflets 20, 22. In some implementations, the gripping members 430 are configured to engage the paddles 420 such that the optional barbed portions 436 engage the valve tissue and the paddles 420 secure the valve repair device 400 to the valve tissue. For example, in certain situations, it can be advantageous to have the paddles 420 maintain an open position and have the gripping members 430 move outward toward the paddles 420 to engage valve tissue and the paddles 420. The paddles 420 and/or the gripping members 430 can be moved between open and closed positions by any means, such as described in FIGS. 24, 25A, and 25B.

In some implementations, the gripping members 430 can be size, shaped, or otherwise configured to approximate the shape and geometry of the native tissue (e.g., leaflets 20, 22) when the device 400 is implanted in the native valve (e.g., mitral valve). For example, the proximal portions of the gripping members 430 (e.g., the portions of the gripping members 430 farthest away from the base 402) can be curved, contoured, or flared outwardly to approximate the shape or geometry of the native tissue (e.g., leaflets 20, 22). As such, when the gripping members 430 engage the native tissue (e.g., leaflets 20, 22), the native tissue can substantially retain its natural shape without additional bending or shearing forces. In the illustrated examples, the paddles 420 are substantially straight. However, the paddles 420 can also be curved, contoured, or flared outwardly to approximate the shape or geometry of the native tissue (e.g., leaflets 20, 22) such that the paddles 420 and the gripping members 430 substantially approximate the shape or geometry of the native tissue (e.g., leaflets 20, 22) when the native tissue is engaged between the paddles 420 and the gripping members 430.

While the example shown in FIG. 32 illustrates a pair of paddles 420 and a pair of gripping members 430, it should be understood that the valve repair device 400 can include any suitable numbers of paddles and gripping members.

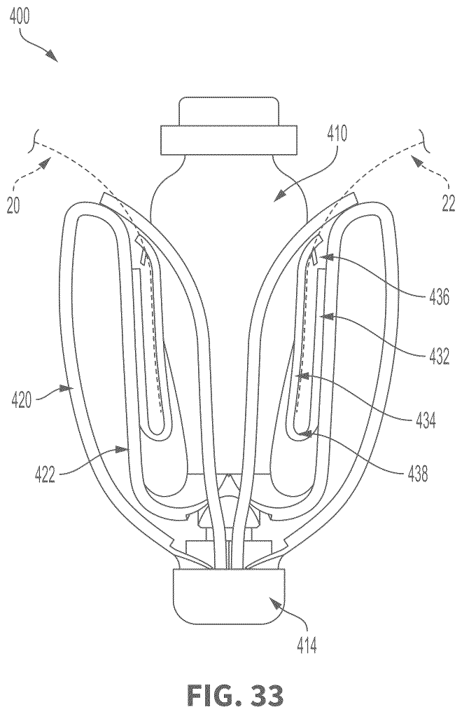

As shown in FIGS. 33-35, in other implementations, the device 400 is an implantable device or implant configured to approximate the shape and geometry of the leaflets 20, 22 of the mitral valve when the device 400 is implanted in the mitral valve. The device 400 can include any features of any implantable device or implant previously disclosed herein, such as the device 100 described in FIGS. 8-21, the device 200 described in FIG. 22, or the device 300 described in FIG. 23, the device 8200 described in FIG. 26-30, and the device 400 can be positioned to engage valve tissue 20, 22 as part of any suitable valve repair system (e.g., any valve repair system disclosed in the present application).

In some implementations, the device or implant 400 includes an optional coaptation portion 404 and an anchor portion 406. The coaptation portion 404 includes a coaptation element 410 that is adapted to be implanted between leaflets of a native valve (e.g., a native mitral valve, native tricuspid valve, etc.) and is slidably attached to an actuation element 412 (e.g., actuation wire, actuation shaft, actuation tube, etc.). The anchor portion 406 includes one or more anchors 408 that are actuatable between open and closed conditions and can take a wide variety of forms, such as, for example, paddles, gripping elements (e.g., clasps), or the like. Actuation of the actuation element 412 opens and closes the anchor portion 406 of the device 400 to grasp the native valve leaflets (e.g., leaflets 20, 22) during implantation. The actuation element 412 (as well as other actuation elements disclosed herein) can take a wide variety of different forms (e.g., as a wire, rod, shaft, tube, screw, suture, line, strip, combination of these, etc.), be made or a variety of different materials, and have a variety of configurations.

The anchor portion 406 and/or the anchors of the device 400 include outer paddles 420 and inner paddles 422 that are, in some implementations, connected between a cap 414 and the coaptation element 410 by portions (e.g., portions 124, 126, 128 described in FIGS. 8-15). The portions can be jointed and/or flexible to move between all of the positions to move between any suitable positions. The interconnection of the outer paddles 420, the inner paddles 422, the coaptation element 410, and the cap 414 by the portions can constrain the positions and movements of the device 400, such as to the positions and movements illustrated in FIGS. 8-15.

In some implementations, the anchor portions 406 and/or anchors 408 can include attachment portions or gripping members. The illustrated gripping members can comprise clasps 430 that include a base or fixed arm 432, a moveable arm 434, optional barbs, friction-enhancing elements, or other securing structures 436 (e.g., protrusions, ridges, grooves, textured surfaces, adhesive, etc.), and a joint portion 438. The fixed arms 432 are attached to the inner paddles 422. In some implementations, the fixed arms 432 are attached to the inner paddles 422 with the joint portion 438 disposed proximate the spacer or coaptation element 410. The joint portion 438 provides a spring force between the fixed and moveable arms 432, 434 of the clasp 430. The joint portion 438 can be any suitable joint, such as a flexible joint, a spring joint, a pivot joint, or the like. In some implementations, the joint portion 438 is a flexible piece of material integrally formed with the fixed and moveable arms 432, 434. The fixed arms 432 are attached to the inner paddles 422 and remain stationary or substantially stationary relative to the inner paddles 422 when the moveable arms 434 are opened to open the clasps 430 and expose the optional barbs, friction-enhancing elements, or other securing structures 436. The clasps 430 can be opened or actuated by any methods described herein. For example, the clasps 430 can be opened by actuation lines which extend from a delivery system (e.g., through a steerable catheter and/or implant catheter).

In some implementations, the gripping members or clasps 430, the fixed arms 432, the moveable arms 434, and/or the joint portions 438 can be size, shaped, or otherwise configured to approximate the shape and geometry of the native tissue (e.g., leaflets 20, 22) when the device 400 is implanted in the native valve (e.g., mitral valve). For example, the distal portions of the moveable arms 434 (e.g., the portions of the moveable arms 434 farthest away from the cap 414 when the moveable arms 434 are in the open position) can be curved, contoured, or flared outwardly to approximate the shape or geometry of the native tissue (e.g., leaflets 20, 22). Additionally, the corresponding portions of the fixed arms 432 (e.g., the portions of the fixed arms 432 closest to the cap 414) can be curved, contoured, or flared to mirror the curve, contour, or flare of the respective moveable arm 434. When the clasps 430 are moved into the closed position and the arms 432, 434 engage the native tissue (e.g., leaflets 20, 22), the native tissue engagement area between the fixed arms 432 and the moveable arms 434 substantially approximates the shape and geometry of the native tissue (e.g., leaflets 20, 22). As such, when the clasps 430 engage the native tissue (e.g., leaflets 20, 22) (FIG. 33), the native tissue can substantially retain its shape.

Further, the contours of the clasps 430, the fixed arms 432, the moveable arms 434, and/or the joint portions 438 can facilitate the deployment or tracking of the device 400 from a delivery system. For example, the contours of the moveable arm 434 can prevent or inhibit the distal portions of the moveable arms 434 and/or the barbs 436 from catching on the delivery device or native tissue during deployment. The contours of the clasps 430 can also improve the elongation of the device 400 in the open position.

While the example shown in FIG. 32 illustrates a pair of clasps 430, including a pair of fixed arms 432 and a pair of moveable arms 434, it should be understood that the implantable device 400 can include any suitable numbers of clasps, fixed arms, and moveable arms.

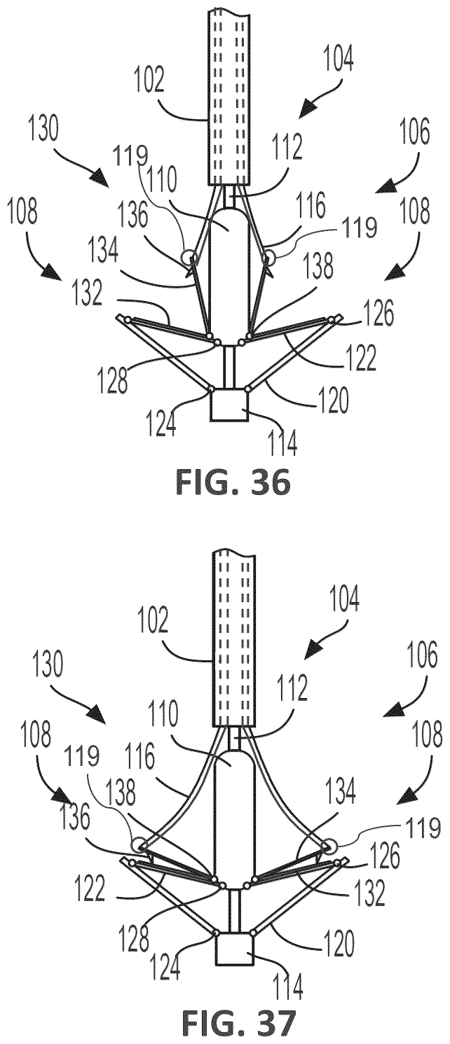

Referring now to FIGS. 36 and 37, one implementation is shown depicting how an implantable device or implant (e.g., an implantable prosthetic device, a prosthetic spacer device, a valve repair device, etc.) is controlled by a delivery system (e.g., a steerable catheter or an implant catheter) during deployment and implantation. In the illustrated implementation, the device 100 of FIGS. 8-15 is deployed from the delivery system 102. However, it will be appreciated that the attachment method can be used for any suitable valve repair system (e.g., any valve repair system disclosed in the present application or the applications cited above) and the delivery system can include one or more of a guide/delivery sheath, a delivery catheter, a steerable catheter, an implant catheter, tube, clasp control elements (e.g., lines, sutures, wires, etc.), combinations of these, etc.

Referring to FIGS. 36-39, one or more actuation elements 116 can extend through the delivery system 102 and be attached to or disposed around a component of the device 100. Each actuation element 116 can be a line, a suture, a tether, a wire, or the like and can comprise a cloth, a polymer, or other bioinert material. The actuation elements 116 can be attached to or coupled to the moveable arms 134 to open and/or close the moveable arms 134 of the device 100. Each actuation element 116 can extend through the delivery system 102 and connect to an actuation loop 119 that is connected to a clasp moveable arm 134. The actuation element 116 can connect loop, tie, or otherwise secure to the actuation loop 119 that is connected to the moveable arm 134). As such, the actuation line 116 can be controlled to open and close the clasps during deployment and implantation.

As described above, the clasps 130 of the device 100 are opened by applying tension to one or more actuation elements 116 attached to the moveable arms 134, thereby causing the moveable arms to articulate, flex, or pivot on the joint portions 138. The clasps 130 can be spring loaded so that in the closed position the clasps 130 continue to provide a pinching force on the grasped native leaflet. Optional barbs, friction-enhancing elements, or other securing structures 136 of the clasps 130 can grab, pinch, and/or pierce the native leaflets to further secure the native leaflets.

As detailed above (e.g., FIGS. 8-15), the paddles 120, 122 can be opened and closed, for example, to press the native leaflets (e.g., native mitral valve leaflets, etc.) between the paddles 120, 122 and/or between the paddles 120, 122 and a spacer or coaptation element 110 (e.g., a spacer, plug, membrane, etc.) by actuating the paddle actuation element 112. Tension can be applied to each of the actuation loops 119 via the actuation element 116 (e.g., line, suture, wire, etc.) to open the respective clasp 130 and tension can be removed from the actuation element 116 to allow the respective clasp 130. The actuation elements 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

In the example illustrated by FIGS. 8-21, after deployment and implantation of the device 100, the clasp actuation lines 116 can be uncoupled from the clasps of the device 100 by pulling one end of each of the actuation lines 116 through the delivery system 102. In some implementations, pulling the actuation line 116 out of the delivery system 102 unflosses or unthreads the actuation line 116 from the attachment portion of the moveable arm 134 and decouples the clasp from the delivery system 102. In some implementations, the actuation line 116 can be pulled through an actuation loop 119 (see FIGS. 36 and 37), thereby uncoupling the actuation line 116 from the actuation loop 119.

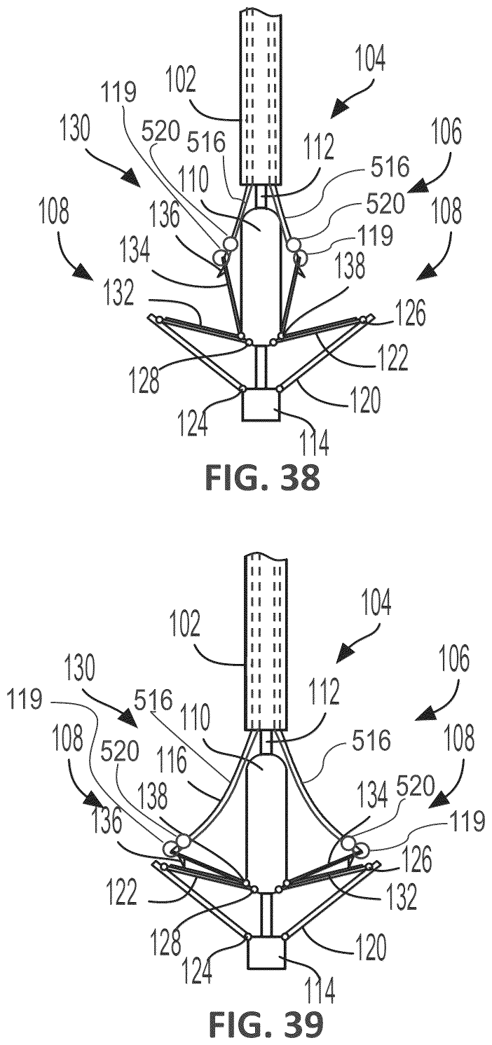

Referring now to FIGS. 38-43B, an example of how a clasp of an implantable device or implant (e.g., an implantable prosthetic device, a prosthetic spacer device, a valve repair device, etc.) is controlled by a delivery system (e.g., a steerable catheter, an implant catheter, clasp control elements, etc.) during deployment and implantation is illustrated. In the illustrated examples, the device 100 of FIGS. 8-15 is deployed from the delivery system 102. However, it will be appreciated that the control and release implementations of FIGS. 38-43B can be used for any suitable valve repair system (e.g., any valve repair system disclosed in the present application or the applications cited herein). The delivery system can include one or more of a guide/delivery sheath, a delivery catheter, a steerable catheter, an implant catheter, tube, and/or clasp control elements combinations of these, etc. For example, the control and release implementations of FIGS. 38-43B can be used with the valve repair device 40256 of FIG. 24 or the implantable valve repair device 8200 of FIGS. 29 and 30, as part of any suitable valve repair system disclosed in the present application or the applications cited above.

In the examples illustrated by FIGS. 38-43B, the device 100 includes an actuation loop 119 on each of the moveable arms 134. The actuation loops 119 is a circle, coil, eyelet, loophole, knot, noose, circle, or other loop of material which is attached to or disposed through a portion of the moveable arm 134. The actuation loops 119 can comprise a cloth, a polymer, and/or a bioinert material. In the illustrated implementation, the actuation loops 119 are disposed at outer ends of the moveable arms 134. However, the actuation loops 119 can be at any suitable position on the moveable arms 134 capable of opening and closing the moveable arms 134 by increasing or decreasing a tension force applied to the actuation loops 119.

In the implementation illustrated by FIGS. 38 through 43B, the actuation elements 116 comprise one or more actuation wires 516 that extend through the delivery system 102. The actuation wires 516 can be a metal wire or similar material capable of conducting an electric voltage and/or current. Each actuation wire 516 includes one or more electrodes 520 at the distal end 518. The electrodes 520 can be capable of generating thermal output from the electric voltage and/or current provided by the actuation wire 516. The distal end 518 of each actuation wire 516 can be affixed or secured to one of the actuation loops 119 of the device 100. The distal end 518 of the actuation wire 516 can be tied, fastened, coupled, or otherwise secured to the actuation loop 119 or each actuation loop 119 can be looped or otherwise fastened around a portion of the distal end 518 of the actuation wire 516.

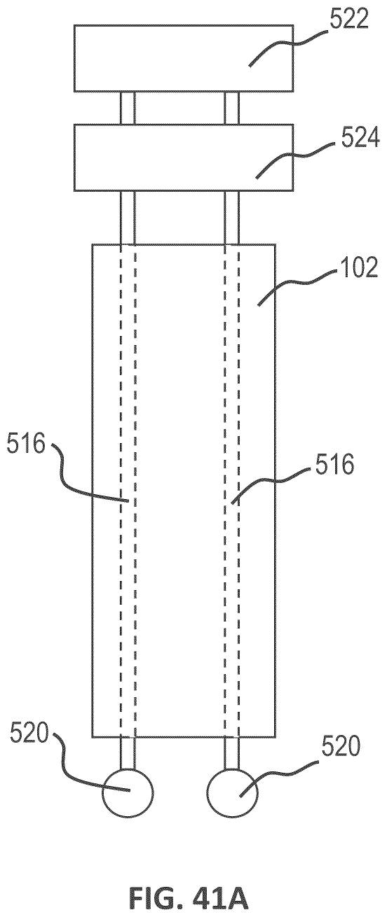

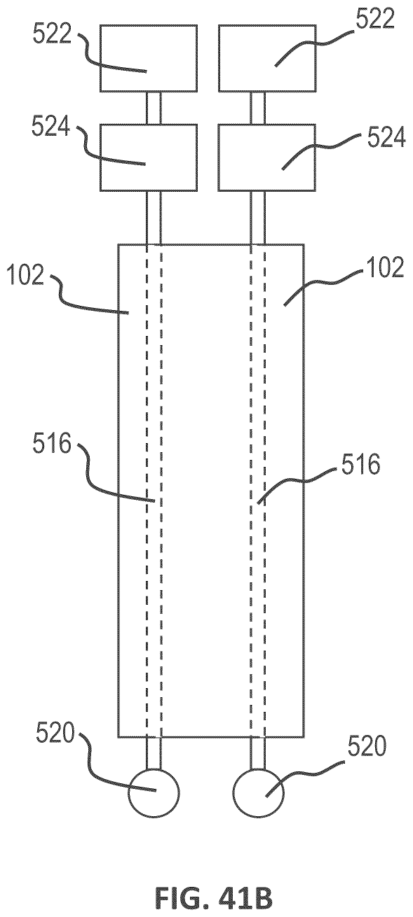

Referring to FIGS. 41A and 41B, one or more energy sources 522 can be attached to the proximal end of each actuation wire 516 and be capable of providing an electrical voltage and/or current, such as a high frequency electrical current, through the actuation wire 516 to the one or more electrodes 520 at the distal end 518 of the actuation wire 516. In some implementations, such as shown in FIGS. 41A, 42A, and 43A, the actuation wires 516 can be connected to a single energy source 522 which is capable of providing an electrical voltage and/or current, such as a high frequency electrical current, through each actuation wire 516 to each electrode 520. In some implementations, such as shown in FIGS. 41B, 42B, and 43B, each actuation wire 516 can be connected to a separate energy source 522 which is capable of providing an electrical voltage and/or current, such as a high frequency electrical current, to the connected actuation wire 516 and the respective electrodes 520. The energy sources 522 can be any suitable device capable of generating and/or providing an electrical voltage and/or current. For example, the energy sources 522 can be batteries, battery packs, power banks, or electric generators, connections to power outlets or sources, or any combination thereof. In some implementations, the proximal end of each actuation wire 516 is also connected to a handle 524 which can control the movement, deployment, and position of the one or more actuation wires 516. In some implementations, such as shown in FIGS. 41A, 42A, and 43A, the actuation wires 516 can be connected to a single handle 524 which can control the movement, deployment, and position of each of the actuation wires 516. In some implementations, such as shown in FIGS. 41B, 42B, and 43B, each actuation wire 516 can be connected to a separate handle 524 which can control the movement, deployment, and position of the connected actuation wire 516. In some implementations, the one or more handles 524 are also capable of controlling the flow of the electrical voltage and/or current from the energy source 522 to the respective actuation wires 516 and electrodes 520.

Referring back to FIGS. 38-40, the moveable arms 134 can be opened and closed by increasing or decreasing a tension applied to the actuation loops 119 via the actuation wires 516 similarly to the actuation lines 116 described in FIGS. 8-15. During deployment from the delivery system 102, the clasps 130 of the device 100 can be opened by applying tension to the one or more actuation wires 516 which transfer the tension force to the actuation loop 119 attached to the moveable arms 134, thereby causing the moveable arms 134 to articulate flex, or pivot on the joint portions 138. The clasps 130 can be spring loaded so that in the closed position the clasps 130 continue to provide a pinching force on the grasped native leaflet. Optional barbs, friction-enhancing elements, or other securing structures 136 of the clasps 130 can grab, pinch, and/or pierce the native leaflets to further secure the native leaflets.

During implantation, the paddles 120, 122 can be opened and closed, for example, to press the native leaflets (e.g., native mitral valve leaflets, etc.) between the paddles 120, 122 and/or between the paddles 120, 122 and a spacer or coaptation element 110 (e.g., a spacer, plug, membrane, etc.). The clasps 130 can be used to grasp and/or further secure the native leaflets by engaging the leaflets with barbs, friction-enhancing elements, or securing structures 136 and pinching the leaflets between the moveable and fixed arms 134, 132. The barbs, friction-enhancing elements, or other securing structures 136 (e.g., barbs, protrusions, ridges, grooves, textured surfaces, adhesive, etc.) of the clasps or barbed clasps 130 increase friction with the leaflets or can partially or completely puncture the leaflets. The actuation wires 516 can optionally be actuated separately so that each clasp 130 can be opened and closed separately, such as by separately actuating each actuation wire 516 with one or more handles 524. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires. After deployment and implantation of the device 100, the actuation wires 516 can remain attached to the actuation loops 119 disposed on the moveable arms 134 of the device 100.

In the illustrated implementation, the device 100 and the actuation wires 516 are both deployed from the same passage (e.g., catheter) of the delivery system 102. However, the device 100 and actuation wires 516 can be deployed in other ways. For example, the device 100 can be deployed from a separate passage from the one or more actuation wires 516, such as from a separate catheter or a separate passage or lumen in the same catheter, or the actuation wires 516 can extend outwardly from radial apertures extending through the side of the delivery system 102 near the distal end of the delivery system 102.

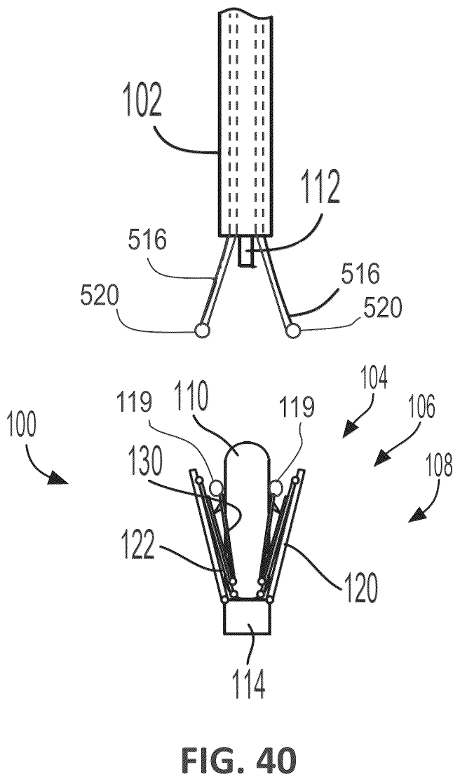

As shown in FIG. 40, after the device 100 has been deployed and implanted, such as in a native valve of a heart, the actuation wires 516 can be decoupled from the device 100. The one or more energy sources 522 can be activated or energized to provide an electrical voltage and/or current to the one or more actuation wires 516. For example, each handle 524 can include a switch which, when activated, permits the flow of electrical current and/or voltage to one or more actuation wires 516. The electrical current and/or voltage can flow through one or more of the actuation wires 516 to energize the one or more electrodes 520. The energized electrodes 520 can then heat, ablate, cauterize, sear, singe or otherwise burn the actuation loops 119 affixed to the moveable arms 134 of the device 100. The energized electrodes 520 can disconnect, sever, or detach the actuation loops 119 such that the actuation wire 516 is decoupled or otherwise disconnected from the device 100. Portions of the actuation loops 119 can remain affixed to the device 100. Each electrode 520 can be energized separately to disconnect, sever, or detach the respective actuation loop 119 or the electrodes 520 can be energized at the same time to disconnect, sever, or detach the actuation loops 119. After each of the actuation wires 516 are decoupled from the device 100, each actuation wire 516, along with the actuation element 112, can be retracted or withdrawn into the delivery system 102 and the delivery system 102 can be retracted from the native valve of the heart. Each actuation wire 516 can be separately decoupled from the device 100 and retracted or withdrawn into the delivery system 102 or all of the actuation wires 516 can be decoupled from the device 100 and retracted or withdrawn into the delivery system 102 at the same time. While the energized electrodes 520 are described as cutting or disconnecting the actuation loops 119 by heat generated from thermal or electrical energy, the energized electrodes 520 can cut or disconnect the actuation loops 119 by other suitable means. For example, the energized electrodes 520 can vibrate at a high frequency, such as ultrasonically, to cut or disconnect the actuation loops 119.

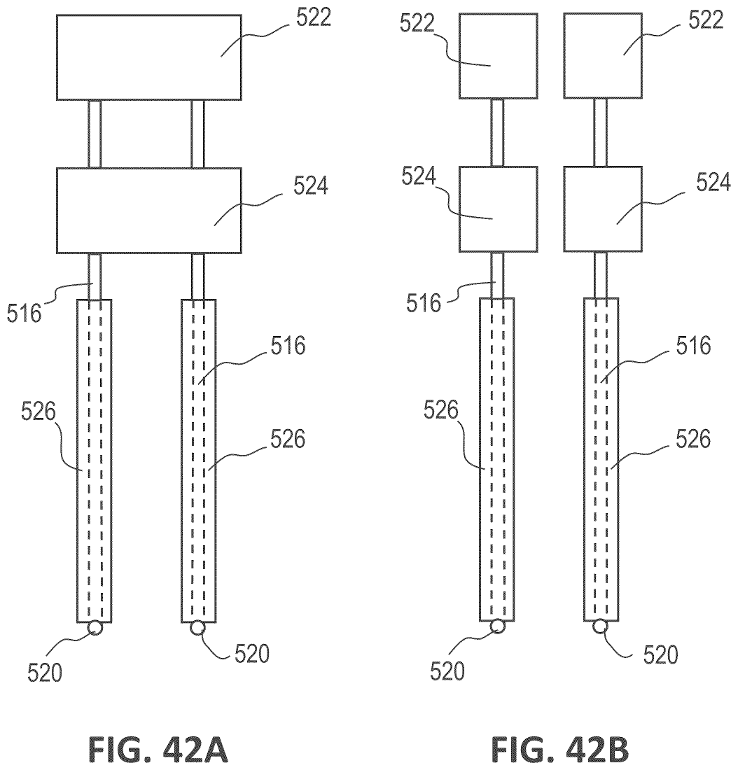

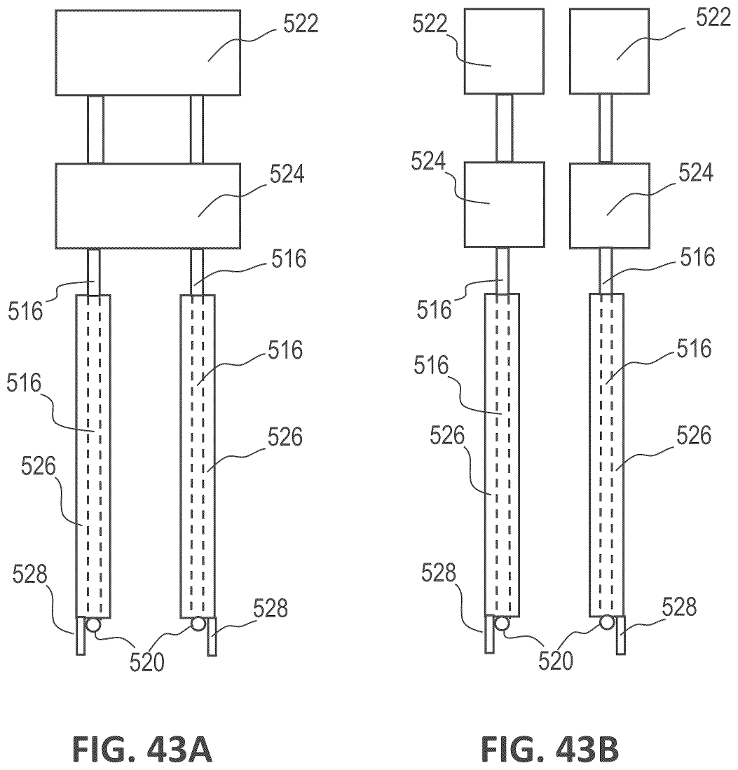

As shown in FIGS. 42A through 43B, each actuation wire 516 can include an insulation 526 disposed around the actuation wire 516 and extending along a length of the actuation wire 516 to the distal end 518. The insulation 526 can prevent or reduce the flow of electrical current and/or voltage and heat radially outward from the actuation wire 516. The insulation 526 can comprise clay, ceramic, porcelain, plastic, rubber, mica, Teflon, polymers such as polytetrafluoroethylene, perfluoroalkoxy, or the like, or any combination thereof.

As shown in FIGS. 43A and 43B, each actuation wire 516 can also include a protector 528 which can prevent or inhibit the actuation wire 516 and/or electrodes 520 from contacting and/or providing heat, vibratory energy, electrical current and/or voltage, etc. to the native tissue of the heart or undesired portions or components of the implantable device or implant. The protector 528 can comprise the same materials as the insulation 526 and prevent or reduce the flow of electrical voltage and/or current or heat from the actuation wire 516 and/or the electrodes 520. In the illustrated implementation, the protector 528 is disposed on a radial side the electrodes 520 and extend distally beyond the electrodes 520. However, the protectors 528 can have any suitable shape, position, or configuration. For example, the protectors 528 can be disposed on multiple sides of the electrodes 520, extend less than the length of the electrodes 520, substantially surround the electrodes 520, curve distally around the electrodes 520, or any combination thereof.

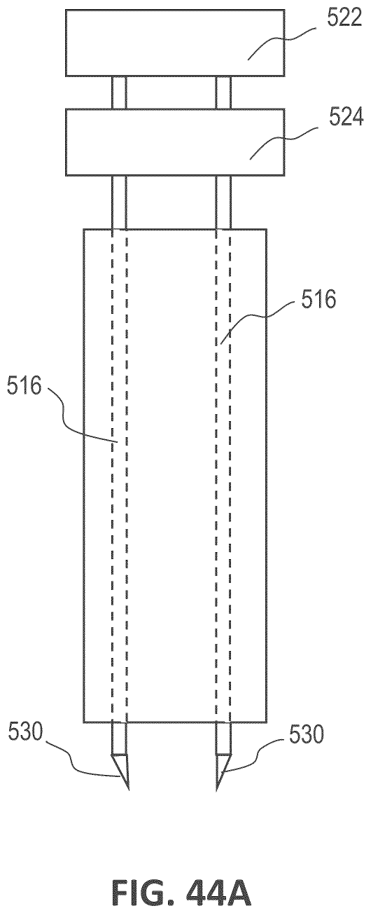

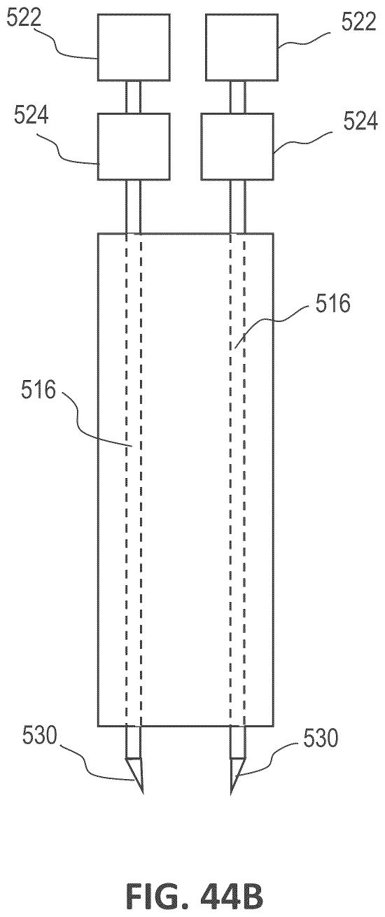

As shown in FIGS. 44A and 44B, the actuation wires 516 can include one or more blades or cutters 530 at the distal end 518 of each actuation wire 516 instead or in addition to the electrodes. The blades or cutters 530 can be sharp and/or serrated to cut or tear actuation loops 119 disposed on the implantable device or implant. The actuation wires 516 can operate substantially similarly to the actuation wires 516 described in FIGS. 38-43 or the cutters can be configured to simply cut the loops 119. In some implementations, such as shown in FIG. 44A, the actuation wires 516 can be connected to a single energy source 522 which is capable of providing an electrical voltage and/or current through the actuation wire 516 and the proximal end of each actuation wire 516 is also connected to a single handle 524 which can control the movement, deployment, and position of each of the actuation wires 516. In some implementations, such as shown in FIG. 44B, each actuation wire 516 can be connected to a separate energy source 522 which is capable of providing an electrical voltage and/or current to the connected actuation wire 516 and the proximal end of each actuation wire 516 is connected to a separate handle 524 which can control the movement, deployment, and position of the connected actuation wire 516. The one or more energy sources 522, such as via the one or more handles 524, can energize the one or more actuation wires 516 and/or actuate the blades or cutters 530 at the distal end of the respective actuation wires 516. The one or more energy sources 522 and the one or more handles 524 can actuate each of the blades or cutters 530 separately or can actuate each of the blades or cutters 530 at the same time. The distal end 518 of each actuation wire 516 can be attached or coupled to the actuation loops 119 of the device 100 (see FIGS. 38-40) and the actuation wires 516 can be used to open and close the clasps 130 of the device 100, as described. In some implementations, one or more of the actuation wires 516 can include an insulation and/or a protector, such as described in FIGS. 42A through 43B.

After the device 100 has been deployed and implanted, such as in a native valve of a heart, the actuation wires 516 can be decoupled from the device. The energy source 522 and/or handle 524 can be activated, energized, moved, or otherwise controlled to cause the cutters 530 to cut, sever, or otherwise disconnect the actuation loops 119 such that the actuation wire 516 is decoupled or otherwise disconnected from the device 100. For example, the energy source 522 can provide electrical current and/or voltage through the actuation wire 516 thereby causing the cutters 530 to actuate, rotate, vibrate, or otherwise move to cut, snip, tear, or sever the actuation loops 119 or the handle 524 can be controlled or operated to cause the cutters 530 to move or rotate to cut, snip, tear, or sever the actuation loops 119. After the actuation wires 516 are decoupled from the device 100, the actuation wires 516 can be retracted or withdrawn into the delivery system 102 and the delivery system 102 can be retracted from the native valve of the heart.

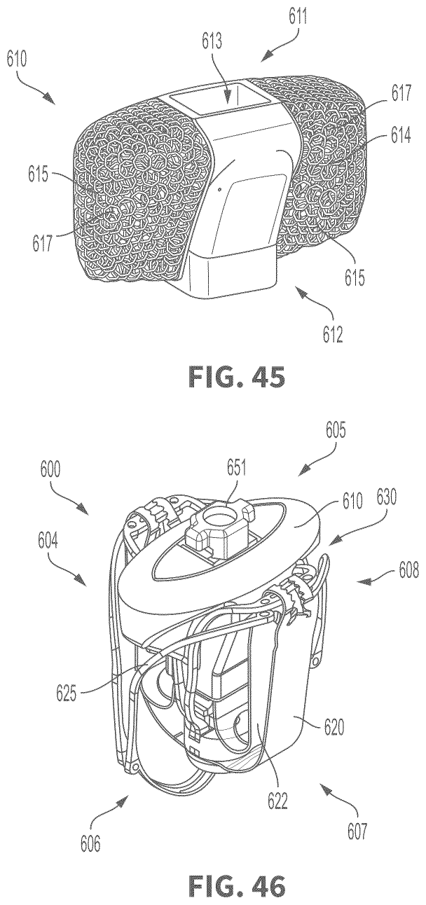

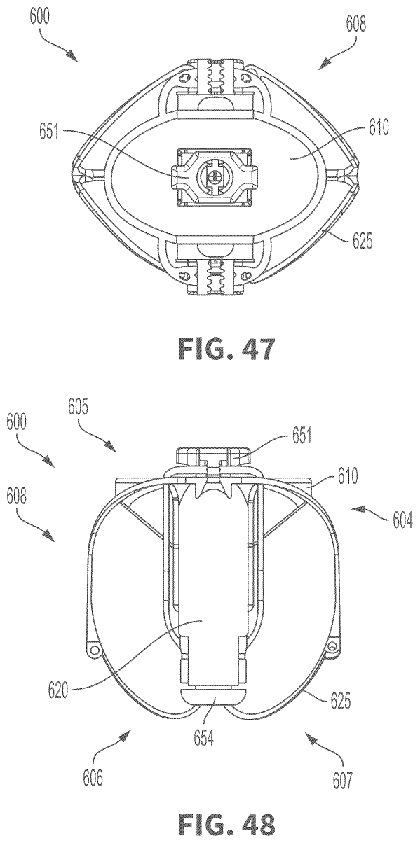

Referring now to FIGS. 45-48, an optional coaptation element 610 can be configured in a variety of ways to be compressible, decrease density, improve visibility, decrease radiopacity, increase volume, increase compliance when deployed in a native heart, and/or to promote ingrowth of tissue once deployed in a native valve of a heart. For example, the coaptation element 610 described above can be compressible such that it fits within, can be moved through, and/or can be deployed from a delivery catheter or the like. The coaptation element 610 can incorporate any of the features previously described herein, such as the coaptation element 610 described in FIG. 4, the coaptation element 110 described in FIGS. 8-21, the coaptation element 210 described in FIG. 22, the coaptation element 310 described in FIG. 23, the coaptation element 3800 described in FIGS. 25A-25B, or the coaptation element 410 described in FIGS. 33-35. Additionally, each of the coaptation elements can be used with any of devices (e.g., 10, 100, 101, 200, 300, 400, 8200, 40256) previously described herein. Each of the coaptation elements can be made of a flexible and/or compressible material such as, for example, rubber, foam, plastic, silicone, or other suitable material or any combination thereof.

The coaptation element 610 extends from a proximal end 611 to a distal end 612 and has a generally elongated and rounded shape. In some implementations, the coaptation element 610 has an elliptical shape or cross-section when viewed from above (FIG. 46) and has a tapered rectangular shape or cross-section when seen from a front view (FIG. 45). A central opening 613 extends through a body portion 614 of the coaptation element 610 from the proximal end 611 to the distal end 612. The central opening 613 has a size that is about the same or is slightly larger than a size of a main shaft or body of an implantable device or implant such that the main shaft can be received within the central opening 613 of the coaptation element 610.

The coaptation element 610 has a plurality of struts 615 disposed throughout the body portion 614 and creating a plurality of gaps or openings 617 in the body portion 614 between the struts 615. The struts 615 and openings 617 can form a lattice structure throughout at least a portion of the body 614 of the coaptation element 610. The struts 615 can be sized, shaped, and oriented in a variety of ways. For example, the struts 615 can be thin and configured to decrease the weight or mass of the coaptation element 610, increase the compressibility of the coaptation element 610, increase the flexibility of the coaptation element 610, and/or to promote tissue ingrowth in the coaptation element 610 when the coaptation element 610 is deployed or otherwise disposed in a native heart. In the illustrated implementation, the struts 615 form a substantially hexagonal lattice structure with openings 617 therebetween. However, it will be appreciated that the struts 615 can be arranged, shaped, oriented, or otherwise configured in any suitable manner to be compressible, decrease density, improve visibility, decrease radiopacity, increase volume, increase compliance when deployed in a native heart, and/or to promote ingrowth of tissue once deployed in a native valve of a heart. For example, the struts 615 can form triangular lattice structures, rectangular lattice structures, circular lattice structures, ovoid lattice structures, any combination of these lattice structures, or any other shaped lattice structures or the struts 615 can extend in a plurality of directions, such as in a non-uniform manner.

The lattice structure of the coaptation element 610 formed by the struts 615 and openings 617 can facilitate deployment of the coaptation element 610 through a delivery catheter while maximizing the volume of the coaptation element 610 when deployed in a native heart. For example, the struts 615 can provide flexible support to the coaptation element 610 such that the coaptation element 610 can be bent or compressed by a compressive force applied to the coaptation element 610 and the openings 617 can decrease the mass of the coaptation element 610 without decreasing the volume of the coaptation element 610. The compressible struts 615 can also increase the engagement or securement of the coaptation element 610 in a native heart as the compressible struts 615 and openings 617 can allow the coaptation element 610 to conform to various spaces and geometries and compensate for anatomical variance. Additionally, the struts 615 can resist plastic deformation such that the struts 615 can return to a normal position when the compressive force is removed.

The lattice structure formed by the struts 615 and openings 617 of the coaptation element 610 can also increase or enhance visibility using imaging technology during delivery and deployment of an implantable device or implant. For example, the lattice structure formed by the struts 615 and openings 617 can increase the echogenicity of the implant and reduce shadowing seen during echocardiography by limiting the overall material density of the coaptation element 610. The lattice structure formed by the struts 615 and openings 617 of the coaptation element 610 can further promote tissue ingrowth when the coaptation element 610 is deployed in a native heart. For example, tissue can grow in and around the openings 617 and struts 615 of the coaptation element 610 when the coaptation element 610 is deployed in the native heart.

A force can be applied to the outside of the body portion 614 of the coaptation element 610 such that the struts 615 compress or bend and the size of the openings 617 between the struts 615 decreases. As such, the overall size of the coaptation element 610 is decreased. In some implementations, any inward force applied to the body portion 614 of the coaptation element 610 can decrease the overall size of the coaptation element 610. However, it will be appreciated that the struts 615 and openings 617 can be configured such that the coaptation element 610 can be compressed in a variety of ways. For example, the struts 615 and openings 617 can be configured such that the coaptation element 610 is only radially compressible from an inward force or that the coaptation element 610 is only longitudinally compressible from an upward or downward force.

The struts 615 can be made from a flexible and/or compressible material such as, for example, foam, rubber, plastic, silicone or other suitable materials or combinations thereof. In some implementations, the struts 615 can be made from a material that can flex or compress when subjected to a force and return to its original shape when the force is removed. The struts 615 can be formed from any suitable method. For example, the struts 615 can be formed from 3D printing, additive manufacturing, additive layer manufacturing, material extrusion, directed energy deposition, powder bed fusion, vat polymerization, injection molding, overmolding, scientific molding, machining, liquid silicone molding, computer numerically controlled machine tooling, laser printing, or any combination of these.

In the illustrated implementation, the entirety of right and left wing portions of the body portion 614 of the coaptation element 610 comprise struts 615 and openings 617 defining a lattice structure and a central portion of the body portion 614 surrounding the central opening 613 is substantially solid. However, it will be appreciated that the coaptation element 610 can have a variety of configurations. For example, in some implementations, the entire body portion 614 of the coaptation element 610 can comprise struts 615 and openings 617 defining a lattice structure. In some implementations, only one or more portions of the coaptation element 610 can comprise struts 615 and openings 617, such as to facilitate deployment of the coaptation element 610, to facilitate the capture and/or engagement of native tissue, and/or to increase the compressibility of the coaptation element 610. For example, the struts 615 and openings 617 can only define a lattice structure in an outer portion or periphery of the body portion 614 such that a core unit made of a harder, more solid material can comprise the center of the body portion 614 to facilitate deployment of the coaptation element 610 through the delivery device while still promoting tissue ingrowth when the coaptation element 610 is deployed in a native heart. In some implementations, the struts 615 and openings 617 may only define a lattice structure in a core of the body portion 614 such that an outer unit made of a harder, more solid material can comprise the outer portion or periphery of body portion 614 to facilitate the capture of leaflet(s) of a native heart valve while decreasing the overall density of the coaptation element 610.

The coaptation element 610 can be sized, shaped, and configured to fit in the native anatomy of the patient that will receive the implant. The coaptation element 610 can be sized, shaped, and configured specifically for the native anatomy of the patient that will receive the implant. The size of the coaptation element 610 can be selected to minimize the number of implants that a single patient will require (preferably one), while at the same time maintaining low transvalvular gradients. In some implementations, the struts 615 and openings 617 of the coaptation element 610 can be sized, shaped, or otherwise configured such that the coaptation element 610 can be compressible and/or expandable to be deployed in any native heart. In some implementations, the surgeon performing the procedure can have a variety of coaptation elements 610 available to him or her during the procedure. The surgeon can select the coaptation element 610 with the desired size, shape, and configuration corresponding to the native anatomy of the patient that will receive the implant. In some implementations, the coaptation element 610 is manufactured specifically for a particular patient that will receive the implant. For example, the size and shape of the patients native valve and/or the area where regurgitation occurs can be measured and the spacer 610 can be manufactured with a size and/or shape that accommodates the measured parameters. That is, each coaptation element can be tailored to any individual patient.

In some implementations, the coaptation element 610 can be removable or otherwise replaceable on the implantable device or implant such that the surgeon performing the procedure can remove one coaptation element 610 from the implantable device or implant and place a coaptation element 610 of size, shape, and configuration desired for the patient on the implantable device or implant. In some implementations, the anterior-posterior distance at the top of the spacer or coaptation element is about 5 mm, and the medial-lateral distance of the spacer or coaptation element at its widest is about 10 mm.

In some implementations, the coaptation element 610 can also include a coating to facilitate the expansion of the coaptation element 610 and/or to facilitate the deployment of the coaptation element 610 from a delivery catheter. The coating can be a polymer or any other suitable material or composition. For example, the coating can be a biocompatible polymer which decreases the friction applied to the coaptation element 610 when the coaptation element 610 is deployed and expanded from a compressed configuration in the delivery catheter.

As shown in FIGS. 46-48, the coaptation element 610 can be attached or incorporated in an implantable device or implant 600. The implantable device 600 is one of the many different configurations that the device 100 that is schematically illustrated in FIGS. 8-14 can take. The device 600 can include any features of any implantable device or valve repair device previously disclosed herein, such as the device 100 described in FIGS. 8-21, the device 200 described in

FIG. 22, the device 300 described in FIG. 23, the valve repair device 40256 described in FIG. 24, the valve repair device 40256 of FIGS. 25A-25B, the device 8200 described in FIG. 26-30, or the devices 400 described in FIGS. 32-35, and the device 600 can be positioned to engage valve tissue 20, 22 as part of any suitable valve repair system (e.g., any valve repair system disclosed in the present application). The device/implant 600 can be a prosthetic spacer device, valve repair device, or another type of implant that attaches to leaflets of a native valve.

The implantable device or implant 600 can be similar in configuration and operation to the implantable device or implants 200, 300 described above. In some implementations, the implantable device or implant 600 includes a coaptation portion 604, a proximal or attachment portion 605, an anchor portion 606, and a distal portion 607. The coaptation element 610 can be included or disposed in the coaptation portion 604 for implantation between leaflets of a native valve. For example, the coaptation element 610 can be disposed on a main shaft of the device 600, such as with the main shaft extending through the central opening 613 of the coaptation element 610. In some implementations, the anchor portion 606 includes a plurality of anchors 608. The anchors 608 can be configured in a variety of ways. In some implementations, each anchor 608 includes outer paddles 620, inner paddles 622, paddle extension members or paddle frames 625, and clasps 630. In some implementations, the attachment portion 605 includes a first or proximal collar 651 (or other attachment element) for engaging with a capture mechanism (e.g., coupler, clamp, tether, etc.) of a delivery system. A delivery system for the device 600 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the paddles 620, 622 are formed from a flexible material that can be a metal fabric, such as a mesh, woven, braided, or formed in any other suitable way or a laser cut or otherwise cut flexible material. The material can be cloth, shape-memory alloy wire—such as Nitinol—, shape memory alloy sheet material—such as National—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 600. In some implementations, the actuation element extends through the proximal collar 651 and the coaptation element 610 to engage a cap 654 of the distal portion 607. The actuation element can be configured to removably engage the cap 654 with a threaded connection, or the like, so that the actuation element can be disengaged and removed from the device 600 after implantation.

The coaptation element 610 extends from the proximal collar 651 (or other attachment element) to the inner paddles 622. The outer paddles 620, inner paddles 622, frames 625, clasps 430, and cap 654 can be formed, shaped, and attached in any manner or configuration described herein, such as similarly to the device 200 described in FIG. 22, the device 300 described in FIG. 23, or the device 8200 described in FIG. 26. The coaptation element 610, outer paddles 620, inner paddles 622, frames 625, and/or clasps 430 can engage native valve tissue (e.g., leaflets 20, 22) by any method discussed in the present application or the applications cited above.

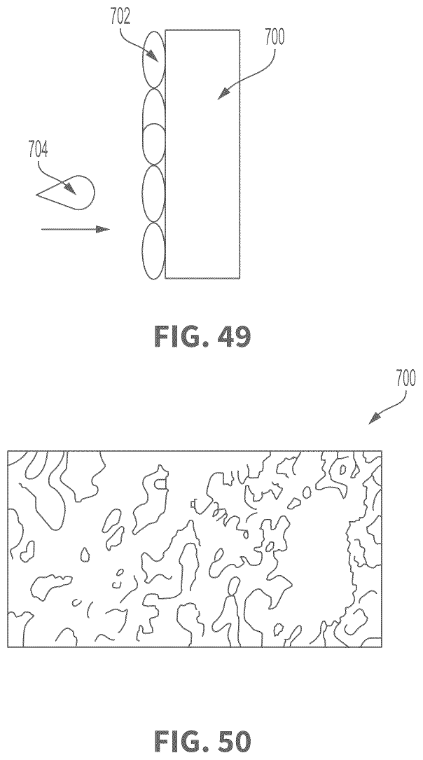

Referring now to FIGS. 49 and 50, one or more surfaces of an implantable device or implant, valve repair device, or valve repair system can be coated or treated to improve the visibility of the device or system during deployment and implantation. For example, one or more portions potions of the device can be coated with a coating, material, or substance to increase or enhance visualization of the device or implant during deployment and implantation, such as when viewed using echocardiographic, sonographic, ultrasound, electromagnetic, or other similar imaging technology. The coating or treatment can be implemented on any of the components of any of the devices (e.g., 10, 100, 101, 200, 300, 400, 600, 8200, 40256) previously described herein or an implantable device or valve repair system having any combination of features disclosed herein.

As shown in FIG. 49, a part or component 700 of the implantable device or valve repair system can be at least partially coated, covered, or topped with a material or coating 702 which can increase or enhance the visibility of the implantable device or valve repair system when viewed using imaging technology. The coating 702 can comprise titanium, hydroxyapatite, ceramic, carbides, silicides, nitrides, cermets, or any combination thereof. The part or component 700 can be any metal surface of the implantable device or valve repair system. For example, the coating 702 can be applied to the joint or connection portions (e.g., 124, 126, 128, 138, 221, 223, 225, 321, 323, 325, 338, 438, 8266), the paddles (e.g., 120, 122, 220, 222, 320, 322, 420, 422, 620, 622, 8256, 40656), the anchors (e.g., 208, 308, 408, 608), the paddle frames (e.g., 224, 324, 625, 8224), the clasps or gripping members (e.g., 130, 230, 330, 430, 630, 40856), the arms (e.g., 132, 134, 332, 334, 432, 434), the coaptation element (e.g., 210, 310, 410, 610, 3800), the collar (e.g., 211, 311, 651), the cap (e.g., 214, 314, 414, 654, 8214), and/or any other suitable component.

The coating 702 can be applied to or deposited on the component 700 using a plasma spray, plasma arc spraying, sputtering, or additive material deposition method to increase the texture of the surface of the component 700, such as by providing a nano-structure to the surface of the component 700. The added texture to the surface of the component 700 can increase visibility of the device or repair system during deployment and implantation as the added texture can increase the effective scattering of the echocardiographic, sonographic, ultrasonic, electromagnetic, or other waveforms used to visualize or image the deployment and implantation of the device or repair system, such as during echocardiographic visualization. For example, the added texture to the surface of the component 700 can reduce the echo or return of the echocardiographic, sonographic, ultrasonic, electromagnetic, or other waveforms used in the visualization or imaging to reduce the ring down and/or shadowing artifacts of the visualization method, thereby permitting a clearer image of the deployment and implantation of the device or repair system.

As shown in FIG. 49, a droplet 704 containing the materials of the coating 702 is formed. The droplet 704 can be formed from a powder, wire, liquid, suspension, or the like which comprises the desired materials of the coating 702. The droplet 704 can be melted, vaporized, ionized, energized, or otherwise heated into a liquid, gas, or plasma. In some implementations, the droplet 704 is heated with a device, such as a plasmatron, plasma torch, or similar device that converts electrical energy into thermal energy which can produce high temperatures, such as temperatures of at least 10,000 K. The liquid, gas, or plasma droplet 704 is then sprayed, propelled, or otherwise deposited onto the surface of the component 700. For example, the droplet 704 can be deposited onto the surface of the component 700 by a plasma jet. The coating 702 can solidify on the surface of the component 700 to form the coating 702 which increases the texture and/or superficial mechanical strength of the surface of the component 700. In some implementations, the droplet 704 can be deposited on the component 700 in a vacuum, such as during high vacuum conditions and/or under an inert gas.

As shown in FIG. 50, an example surface of a component 700 is shown after the coating 702 has been applied. The coating 702 increases the texture of the surface of the component 700 thereby decreasing the smoothness of the surface of the component 700. For example, the coating 702 can include lamella, plates, flakes, or similar particles of material which increase the texture of the surface of the component 700. When the surface of the component 700 is subjected to echocardiographic, sonographic, ultrasound, electromagnetic, or other similar imaging technology, the waveforms scatter when they echo or reflect, thereby decreasing the amount and/or intensity of the waveforms which echo back to the imaging device. As such, the coating 702, when applied to an implantable device or valve repair system, can increase the visibility of the device repair system when viewed using echocardiographic, sonographic, ultrasound, electromagnetic, or other similar imaging technology by decreasing the intensity of the reflected waveforms and providing a clearer image of the implantable device or valve repair system.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A method for implanting a valve repair device, the method comprising:
deploying the valve repair device from a delivery system such that one or more lines extend from the valve repair device to the delivery system;
actuating the valve repair device to attach the valve repair device to leaflets of a native heart valve; and
applying energy to sever at least one of the one or more lines.

2. The method of claim 1 wherein the actuating comprises applying a tension force to an actuation wire coupled to an actuation loop of the valve repair device to open a clasp of the valve repair device, and lessening the tension force to close the clasp.

3. The method of claim 2 wherein the applying energy comprises energizing an electrode at a distal end of the actuation wire to decouple the actuation wire from the actuation loop.

4. The method of claim 2, wherein the actuation wire includes an insulation.

5. The method of claim 3, wherein the electrode is energized by an energy source.

6. The method of claim 3, wherein a handle controls a flow of energy to the electrode.

7. The valve repair device of claim 1 further comprising sterilizing the valve repair device.

8. A valve repair device for repairing a native valve of a patient, the valve repair device comprising:
a pair of paddles;
a pair of contoured clasps moveable between an open position and a closed position; and
wherein each contoured clasp of the pair of contoured clasps approximates a shape of the native valve when the valve repair device is deployed in the native valve and the pair of contoured clasps are in the closed position.

9. The valve repair device of claim 8, wherein each contoured clasp includes a contoured moveable arm and a fixed arm.

10. The valve repair device of claim 9, wherein the fixed arm has a contour which mirrors the contoured moveable arm.

11. The valve repair device of claim 8, wherein each paddle includes an inner paddle and an outer paddle.

12. The valve repair device of claim 8, further comprising a coaptation element disposed between the pair of contoured clasps.

13. The valve repair device of claim 12, wherein the coaptation element includes a plurality of struts and a plurality of openings defining a lattice structure.

14. The valve repair device of claim 8, further comprising a coating which decreases a number of waveforms which echo back when using an imaging technology.

15. The valve repair device of claim 14 wherein the coating comprises a plasma coating.

16. A valve repair device for repairing a native valve of a patient, the valve repair device comprising:
an anchor portion including a pair anchors;
a coaptation portion including a coaptation element;
wherein each anchor includes an outer paddle and an inner paddle; and
wherein the coaptation element includes a body portion with a plurality of struts and a plurality of openings defining a lattice structure.

17. The valve repair device of claim 16, wherein the lattice structure defines wing portions of the body portion of the coaptation element.

18. The valve repair device of claim 16, wherein the lattice structure defines substantially an entirety of the body portion of the coaptation element.

19. The valve repair device of claim 16, wherein the plurality of struts compress when subjected to a force applied to the body portion of the coaptation element.

20. The valve repair device of claim 16, further comprising a coating which decreases a number of waveforms which echo back when using an imaging technology.
